(19) United States
(12) Patent Application Publication
(10) Pub. No.: US 2006/0182791 A1
(43) Pub. Date: Aug. 17, 2006

(54) Transdermaltherapeutic system containing a pramipexol active agent

(76) Inventors: Frank Theobald, Bad Breisig (DE); Wolfgang Laux, Diez (DE); Beatrix Platt, Hausten (DE); Regine Kaufmann, Neuwied (DE)

Correspondence Address: Cathy R Moore;Propat, 425 C South Sharon Amity Road, Charlotte, NC, 28211, US

(21) Appl. No.: 10/564,932

(22) PCT Filed: Jul. 14, 2004
(86) PCT No.: PCT/EP04/07770

§ 371 Date: Jan. 13, 2006

(30) Foreign Application Priority Data
Jul. 23, 2003 (DE) 103 33 393.2

(51) Int. Cl. A61K 31/428; A61K 9/70

(52) U.S. Cl. 424/449; 514/367

(57) ABSTRACT

The invention relates to a transdermal therapeutic system (TTS) releasing an active pramipexol agent during a time ranging from 4 to 7 hours.

The present invention relates to a transdermal therapeutic system (TTS) for administering pramipexol. It relates in particular to a self-adhesive pramipexol TTS which is able to deliver the active ingredient pramipexol as base continuously over a prolonged period of, preferably, 4 to 7 days to a person who depends on a continuous supply of an effective amount of this active ingredient.

A transdermal therapeutic system (TTS) is a pharmaceutical dosage form which has a layered structure and consists of at least one active ingredient-containing polymer layer and one backing layer which is ordinarily impermeable for the active ingredient. The TTS may also optionally comprise further layers, frequently for example a membrane which controls the rate of release of the active ingredient, a pressure-sensitive adhesive layer which ensures adhesion of the TTS to the patient's skin, a barrier layer, and a protective layer which covers the active ingredient-delivering side of the TTS until use. In a TTS with a particularly simple structure, the active ingredient-containing polymer layer is itself provided with a pressure-sensitive adhesive so that it is possible to dispense with an additional pressure-sensitive adhesive layer, an adhesive ring which for example encloses a circular reservoir, or an additional pressure-sensitive adhesive top plaster (covering plaster). A TTS is able, owing to its constructional elements, to deliver the active ingredient continuously and in controlled manner to the patient's skin. After passing through the various outer layers of skin, the active pharmaceutical ingredient is taken up by the underlying blood vessels. The continuous delivery results in particularly uniform plasma levels. Transdermal administration also entails the advantage of avoiding the gastrointestinal tract.

The active ingredient pramipexol has the chemical name (S)-2-amino-4,5,6,7-tetrahydro-6-(propylamino)benzothiazole. The active ingredient is thus in chemical terms a base. It has the CAS Registry No. [104632-26-0] and is regarded as the first non-ergotic, presynaptic dopamine D₂ agonist. The active ingredient is obtainable in the form of the hydrochloride as tablet under the proprietary names Sifrolo® and Mirapex®. As such, it is employed as anti Parkinson agent and for the treatment of extrapyramidal disorders. Pramipexol is used for idiopathic (without detectable cause, quasi self-originated) Parkinson's disease both in the early stage and in the advanced stage, and in this case in combination with levodopa.

The chemical formula of pramipexol is:

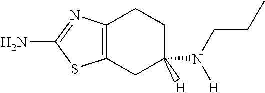

When Parkinson's disease is treated with pramipexol which can be administered orally it is necessary first to establish for each patient the individual dose which is adjusted optimally for efficacy and tolerability. This dose titration usually takes place at weekly intervals, administering in the first week an amount of pramipexol hydrochloride equivalent to 0.088 mg of pramipexol base three times a day. In the second week, an amount of pramipexol hydrochloride equivalent to 0.18 mg of pramipexol base is administered three times a day. In the third week, finally, an amount of pramipexol hydrochloride equivalent to 0.36 mg of pramipexol base is administered three times a day.

Once the individual dose has been adjusted in this way, the average daily dose generally corresponds to 1.5 mg of pramipexol hydrochloride, meaning oral administration of 0.36 mg of pramipexol base three times a day.

Parkinson's disease means a disorder of the basal ganglia which is characterized in particular by impairments of movement.

Besides the treatment of Parkinson's disease, pramipexol is also employed for the treatment of so-called restless leg syndrome; compare DE 197 01 619 A1, which is incorporated herein by reference.

The prior art includes transdermal therapeutic systems (TTS) with the active ingredient pramipexol, especially its (−) enantiomer and pharmaceutically acceptable acid addition salts. Thus, EP 428 038 A2 describes transdermal therapeutic systems with an active ingredient reservoir composed of an emulsion-polymerized polyacrylate and 5 to 30% by weight of the active ingredient pramipexol. The carrier material preferably employed is Eudragit NE 30 D® from Röhm GmbH Darmstadt. This product is obtainable in the form of an aqueous dispersion of a copolymer of neutral character based on ethyl acrylate and methyl methacrylate with a dry matter content of 30%. The average molecular weight is 800 000. Active ingredient-containing sheets can be produced from Eudragit NE 30 D® but do not adhere pressure-sensitively. The active ingredient-containing reservoirs in particular embodiments of these TTS have an area of 20 cm², a thickness of 200 μm and an active ingredient content of 9% by weight. The active ingredient-containing reservoirs which were provided with a covering plaster for attachment to the skin were able to deliver a daily dose of about 2.5 mg over a period of 3 and 4 days respectively to two subjects. In vitro investigations on samples of these TTS show that about 70% of the amount of active ingredient had been delivered after only 4 days and that only about a further 10% of the amount of active ingredient originally present in the reservoir can be released in the subsequent three days.

U.S. Pat. No. 6,465,004 B1 discloses a transdermal therapeutic system which, besides the active pharmaceutical ingredient and one or more adhesives, comprises cellulose acetate butyrate as constituent which is insoluble in water but soluble in the adhesive. The latter is an esterified cellulose derivative intended to prevent crystallization of the active ingredient in the pressure-sensitive adhesive. Pramipexol is also considered as an active pharmaceutical ingredient. However, it is not disclosed whether a pramipexol TTS with a corresponding structure is suitable for continuous administration of the active ingredient over a prolonged period of, preferably, 4 to 7 days.

German published specification DE 100 33 853 A1 discloses transdermal therapeutic systems which, besides the active pharmaceutical ingredient (including pramipexol) and a matrix material, comprise colloidal silicon dioxide as further constituent. A pramipexol TTS able to administer an effective amount of this active ingredient continuously over a prolonged period of, preferably, 4 to 7 days is not disclosed.

It is an object of the present invention to provide a self-adhesive transdermal therapeutic system (TTS) which—after establishment of an individual daily dose—delivers the active ingredient pramipexol continuously to the patient in the long-term therapy phase without the need for administration of an oral tablet three times a day. It is also intended for the active ingredient-containing polymer layer or the side of the TTS facing the skin to have a pressure-sensitive adhesive finish so that it is possible to dispense with the use of an additional pressure-sensitive adhesive top plaster for fixing to the skin. It is preferably intended that the administration of a transdermal therapeutic system take place in this long-term phase in such a way that the patient is supplied adequately with active ingredient for a prolonged period, preferably for 4 to 7 days.

The object is achieved by a transdermal therapeutic system (TTS) with the active ingredient pramipexol which delivers the active ingredient over a prolonged period, which is preferably 4 to 7 days, continuously to a person requiring the active ingredient pramipexol.

Such a TTS comprises a—preferably active ingredient-impermeable —backing layer, at least one active ingredient-containing layer and a protective layer to be removed before use, where the active ingredient-containing layer comprises the active ingredient pramipexol. The term pramipexol means in the context of the present invention the S-(−) enantiomer, and the R-(+) enantiomer and a—preferably racemic—mixture of these two enantiomers, preferably the S-(−) enantiomer. In these forms, pramipexol can be present in the at least one active ingredient-containing layer as free base, as hydrate, solvate or pharmaceutically acceptable salt (e.g. as hydrochloride). It is particularly preferred to use pramipexol as S-(−) enantiomer in the form of the free base.

The active ingredient-containing layer further comprises a pressure-sensitive adhesive which is able to attach the TTS securely to a single site on the user's skin throughout the application period of, preferably, 4 to 7 days. The TTS may also comprise further layers, for example a membrane controlling the rate of release of the active ingredient, at least one additional active ingredient-containing layer, at least one supporting layer to increase the mechanical stability of the TTS, and a pressure-sensitive adhesive layer located on the side of the TTS facing the skin.

Pressure-sensitive adhesives which are suitable for the active ingredient-containing layer and, if appropriate, the pressure-sensitive adhesive layer located on the side of the TTS facing the skin are derived from the group of silicones, polyisobutylenes and polyacrylates. Polyacrylates (acrylate pressure-sensitive adhesives) without carboxyl groups have proved to be particularly suitable.

Likewise suitable were silicone pressure-sensitive adhesives (e.g. Dow Corning Bio-PSA Q7-4301), pressure-sensitive adhesives based on polyisobutylene/polybutene (PIB/PB) and combinations of styrene-isoprene-styrene block copolymers in combination adhesive resins.

The active ingredient-containing layer may consist of a single, preferably homogeneous, active ingredient-containing pressure-sensitive adhesive layer, but may also be composed of two or more layers which differ in polymer and active ingredient composition. The pressure-sensitive adhesive layer may also be composed of a mixture of two or more different pressure-sensitive adhesives.

Polyacrylates are generally prepared by polymerizing various monomers (at least one monomer from the group comprising acrylic acid, methacrylic acid, acrylic esters and methacrylic esters, where appropriate together with vinyl acetate) and in particular from mixtures thereof. Solvents used in the polymerization to prepare a suitable polyacrylate are preferably organic solvents, in some cases also water.

Depending on the structure of the monomers employed in the polymerization, the resulting polyacrylates may comprise functional groups. Widely used polyacrylates have —OH groups (hydroxyl groups) or —COOH groups (carboxyl groups) as functional groups. Hydroxyl group-containing polyacrylates are obtained on use of hydroxyl group-containing acrylic esters and/or hydroxyl group-containing methacrylic esters as sole monomer or as constituent in the monomer mixture. Carboxyl group-containing polyacrylates are produced when acrylic acid and/or methacrylic acid are used as monomer or in the monomer mixture. Carboxyl group-free polyacrylates are therefore those prepared from a monomeric (meth)acrylic acid derivative or a corresponding monomer mixture without use of acrylic acid or methacrylic acid.

The hydroxyl group-containing polyacrylates include for example Durotak 2287 whose monomer composition is, according to WO 96/40087, vinyl acetate, 2-ethylhexyl acrylate, hydroxyethyl acrylate and glycidyl acrylate and which is produced by National Starch. This polyacrylate has proved to be a stable and well-tolerated pressure-sensitive adhesive polymer for producing transdermal therapeutic systems.

It has now emerged, surprisingly, that pressure-sensitive adhesives from the group of polyacrylates which are able to take up pramipexol in sufficient amount and satisfy the desired requirements of controlled release over a prolonged period of, preferably, 4 to 7 days are in particular those free of carboxyl groups. It is unnecessary to add excipients to generate pH-controlled conditions on the skin (e.g. a weak acid, a weak base or inorganic or organic salts which form a buffer system on the skin), crystallization inhibitors or colloidal silicon dioxide in a penetration-promoting amount to the matrix. These pressure-sensitive adhesives from the group of polyacrylates are in this connection produced exclusively by polymerization in an organic solvent or solvent mixture—not in water or an aqueous dispersion.

Thus, suitable polyacrylates are polymers (homopolymers, copolymers and block copolymers) which can be prepared from monomers of the group comprising acrylic esters, methacrylic esters and mixtures thereof, where appropriate with additional vinyl acetate.

The most suitable acrylic esters and methacrylic esters are in this connection those having linear, branched or cyclic aliphatic C₁-C₁₂ substituents without other functional groups. This group includes in particular n-butyl acrylate, n-butyl methacrylate, ethyl acrylate, 2-ethylhexyl acrylate, ethyl methacrylate, methyl acrylate, methyl methacrylate, tert-butyl acrylate, sec-butyl acrylate, tert-butyl methacrylate, cyclohexyl methacrylate, 2-ethylhexyl methacrylate, isobornyl methacrylate, isobutyl methacrylate, isopropyl acrylate and isopropyl methacrylate. Particular preference is given to 2-ethylhexyl acrylate and methyl acrylate.

However, it is also possible for the monomer mixture used to prepare the polyacrylate to comprise acrylic esters and methacrylic esters having functional groups. By these are meant primarily hydroxyl group-containing esters, that is to say 2-hydroxyethyl acrylate, 2-hydroxyethyl methacrylate, 3-hydroxypropyl acrylate and 3-hydroxypropyl methacrylate. However, substances such as acrylamide, dimethylaminoethyl acrylate, etc. can also be regarded in the sense of this description as acrylic esters and methacrylic esters comprising functional groups.

The proportion of acrylic esters and methacrylic esters comprising such functional groups in the monomer mixture should in this connection be less than or equal to 10% by weight. The proportion of acrylic esters comprising functional groups and methacrylic esters comprising functional groups in the monomer mixture is preferably less than 2% by weight. In a preferred embodiment, the proportion of acrylic esters comprising functional groups and methacrylic esters comprising functional groups in the monomer mixture is less than 0.2% by weight. A particularly preferred monomer mixture is one comprising no acrylic esters and methacrylic esters comprising functional groups.

As already mentioned, however, vinyl acetate can also be used as comonomer together with at least one monomer from the group of acrylic esters and methacrylic esters for preparing the polyacrylate. The proportion of vinyl acetate in the monomer mixture used to prepare this polyacrylate should be below 50% by weight, preferably below 25% by weight. A vinyl acetate content between 0 and 5% by weight is particularly preferred.

The proportion of pramipexol in the form of the base in dissolved, emulsified or dispersed form in one of the abovementioned pressure-sensitive adhesives can be less than 75% by weight. It is preferably in the range between 2 and 40% by weight and a range between 10 and 25% by weight is particularly preferred. However, the optimal loading of the pressure-sensitive adhesive with active ingredient also depends on the specific requirements relating to the desired timing of release, on the presence of further constituents in the active ingredient-containing pressure-sensitive adhesive layer and on the physicochemical conditions present thereby. If the active ingredient pramipexol is present as dispersion in the active ingredient-containing layer, the solid particles of the active ingredient preferably have a size of less than 20 μm.

The transdermal therapeutic systems may comprise one or more solvents to improve dissolving of the active ingredient in the polymer. Suitable for this purpose are propylene glycol, ethyl oleate, 1,2-propanediol, 1,3-butanediol, Transcutol, propylene glycol monocaprylate, Solketal, oleic acid, 1-methylpyrrolidone, glycerol, lauryl lactate, triacetin, glycerol monooleate, sorbitan monooleate and sorbitan trioleate. Propylene glycol, butanediol and lauryl lactate are particularly preferred.

The TTS may comprise antioxidants to increase the stability, e.g. ascorbic acid, esters of ascorbic acid, sodium EDTA, bisulfite, etc., which may preferably be present in a proportion by weight of up to 1% in the active ingredient-containing layer. Storage of the TTS in an air-tight primary packaging (blister pack, side-sealed bag) under a protective gas atmosphere (N₂, Ar, etc.) also increases the stability.

The approved maximum daily dose of pramipexol, based on pramipexol base, in the therapy of Parkinson's disease is 3.2 mg per day. Based on a transdermal therapeutic system which is to have an area of 20 cm² for delivering the active ingredient pramipexol to the skin, the necessary flux rate resulting therefrom is 6.25 μg/cm² h.

A particularly preferred transdermal therapeutic system is able to deliver pramipexol with a flux rate greater than 5 μg/cm² h over the period from 8 hours after application to 72 h after application.

Pramipexol can be used by means of the transdermal therapeutic system described herein for the therapeutic treatment or for minimizing the symptoms of depression, tremor, ADHD (attention deficit hyperactivity disorder), anhedonia, HIV dementia, drug dependency and schizophrenia. It is preferably employed for the treatment of ALS (amyotrophic lateral sclerosis), adiposity, obesity and diabetes and, because of its neuroprotective effect and its anticonvulsant effect. The pramipexol-containing TTS is particularly preferably employed for restless leg syndrome and for Parkinson's disease.

The following examples should explain the present invention in more detail without them being regarded as restriction to these cases.

### EXAMPLE 1

A mixture is prepared from 10% by weight of pramipexol (as base), 20% by weight of butanediol and 70% by weight of Durotak 2287 and is spread by knife application onto a support sheet serving as later backing layer to give—after drying—a pressure-sensitive adhesive layer with a basis weight of 200 g/m². TTS samples which can be employed for in vitro investigations are cut out of the two-layer laminate of backing layer and active ingredient-containing pressure-sensitive adhesive layer obtained in this way.

### EXAMPLE 2

A TTS consisting of backing layer and two active ingredient-containing layers is produced. The first active ingredient-containing layer (reservoir layer) consists of 40% by weight of pramipexol (base) and 60% by weight of Durotak 2287 and has a basis weight of 100 g/m². The second active ingredient-containing layer (pressure-sensitive adhesive layer) consists of 3% by weight of pramipexol (base) and 97% by weight of Durotak 2287 and has a basis weight of 30 g/m². TTS samples for the in vitro investigations are cut out of the laminate consisting of backing layer, reservoir layer and pressure-sensitive adhesive layer obtained in this way.

### EXAMPLE 3

The pramipexol flux across human full-thickness skin were determined in vitro for the two TTS samples of examples 1 and 2.

The in vitro investigations were carried out with a modified Franz cell. Human full-thickness skin derived from plastic surgery served as membrane. The TTS area was 1.54 cm². A phosphate buffer solution of pH 7.4 mixed with 0.1% sodium azide was used as acceptor solution. The acceptor volume was 9 ml and was removed completely after 24, 32, 48, 56 and 72 hours and replaced by new buffer solution. The Franz cells were located in a water bath whose temperature was set at 32° C. The pramipexol content in the phosphate buffer solution was determined by suitable HPLC analyses.

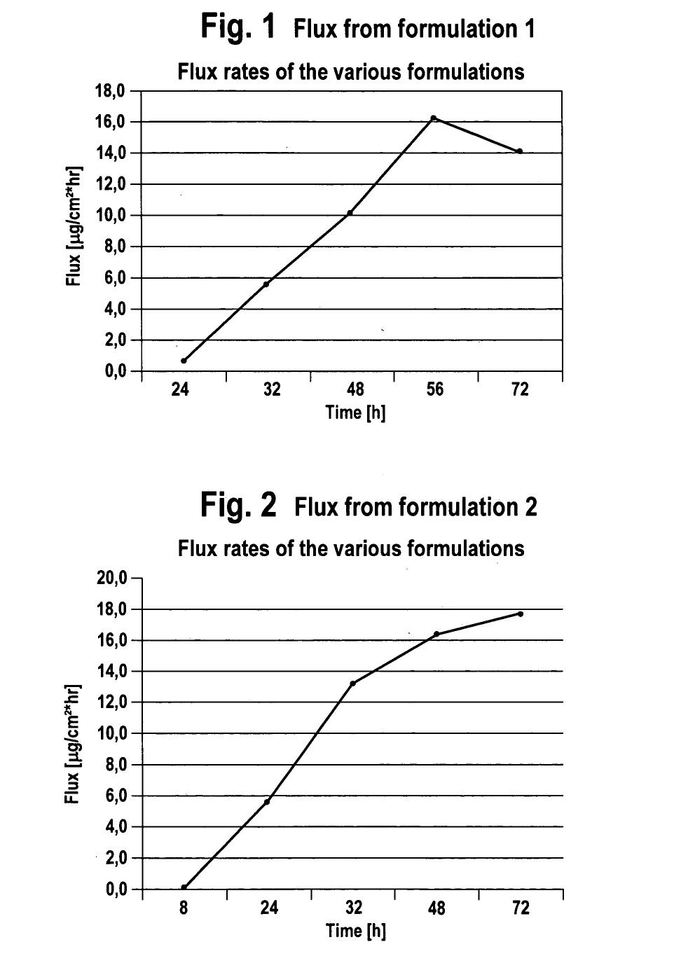

The results are detailed in FIGS. 1 and 2. It was possible to show by these in vitro investigations on human full-thickness skin that TTS formulations comprising at least one active ingredient-containing layer with 10 to 40% by weight of pramipexol in the form of the base are suitable for continuous transdermal administration of this active ingredient for up to 7 days.

Adhesives having carboxyl functions as functional groups in the polymer (e.g. Durotak 2051 or Durotak 2353), i.e. those produced using acrylic acid or methacrylic acid, proved to be unsuitable for production.

1. A transdermal therapeutic system for continuous administration of pramipexol comprising a backing layer and at least one active ingredient-containing polymer layer which comprises the active ingredient pramipexol, wherein the active ingredient-containing polymer layer comprises at least one pressure-sensitive adhesive polymer selected from the group of silicones, polyisobutylenes, polybutenes, styrene-isoprene-styrene block copolymers in combination with resins, and of carboxyl group-free polyacrylates, where the active ingredient pramipexol is present therein in a proportion of between 10 and 40% by weight.

2. The transdermal therapeutic system as claimed in claim 1, which comprises a further pressure-sensitive adhesive layer, an additional membrane which controls the rate of release of pramipexol, an additional active ingredient-containing layer or an additional supporting layer.

3. The transdermal therapeutic system as claimed in claim 1, wherein the pressure-sensitive adhesive polymer is a carboxyl group-free polyacrylate which can be prepared by polymerization of a monomer mixture of at least one acrylic ester or methacrylic ester.

4. The transdermal therapeutic system as claimed in claim 3, wherein the monomer mixture comprises at least one acrylic ester or methacrylic ester with linear, branched or cyclic aliphatic C₁-C₁₂ substituents without other functional groups.

5. The transdermal therapeutic system as claimed in claim 3 4, wherein the monomer mixture additionally comprises at least one hydroxyl group-containing acrylic ester or one hydroxyl group-containing methacrylic ester in a proportion by weight of less than 10%.

6. The transdermal therapeutic system as claimed in claim 3, wherein the monomer mixture additionally comprises vinyl acetate in a proportion by weight of less than 50%.

7. The transdermal therapeutic system as claimed in claim 1, wherein the active ingredient pramipexol is present in the active ingredient-containing polymer layer in dissolved, emulsified and/or dispersed form.

8. The transdermal therapeutic system as claimed in claim 1, wherein the active ingredient pramipexol is present as S-(−) enantiomer, R-(+) enantiomer or racemic mixture of these two enantiomers in the active ingredient-containing polymer layer.

9. The transdermal therapeutic system as claimed in claim 1, wherein the active ingredient pramipexol is present as a free base, hydrate, solvate and/or pharmaceutically acceptable salt in the active ingredient-containing polymer layer.

10. The transdermal therapeutic system as claimed in claim 1, wherein the active ingredient pramipexol is present as S-(−) enantiomer in the form of a free base in the active ingredient-containing polymer layer.

11. The transdermal therapeutic system as claimed in claim 1, which is able to deliver wherein said transdermal therapeutic system delivers the active ingredient pramipexol continuously to a patient's skin over a period of from 4 to 7 days.

12. The transdermal therapeutic system as claimed in claim 1, which is able to release the active ingredient pramipexol with a flux rate greater than 5 μg/cm² h over the period between 24 hours after administration to 168 h after administration.

13. The transdermal therapeutic system as claimed in claim 1, which is able to release said transdermal therapeutic system releasing the active ingredient pramipexol with a flux rate greater than 5 μg/cm² h over the period between 24 hours after administration to 72 h after administration.

14. The transdermal therapeutic system as claimed in claim 1, wherein the active ingredient pramipexol is present therein in a proportion of between 10 and 25% by weight.

15. The transdermal therapeutic system as claimed in claim 1, wherein the daily delivery rate of pramipexol is between 0.1-10 mg.

16. The transdermal therapeutic system as claimed claim 6, wherein said vinyl acetate is present in a proportion of less than 25% by weight.

17. The transdermal therapeutic system as claimed claim 15, wherein the daily delivery rate of pramipexol is between 0.5 to 4.5 mg.
